(19) United States
(12) Patent Application Publication
(10) Pub. No.: US 2023/0011195 A1
(43) Pub. Date: Jan. 12, 2023

(54) ARTICULAR FRACTURE REHABILITATION PROCESS MEASURING DEVICE

(71) Applicant: BOHONG SU, San Jose, CA (US)

(72) Inventor: BOHONG SU, San Jose, CA (US)

(21) Appl. No.: 17/369,117

(22) Filed: Jul. 7, 2021

(51) Int. Cl. A61B 5/11; A61B 5/00

(52) U.S. Cl. CPC A61B 5/1121; A61B 5/7405; A61B 5/742; A61B 5/4842; A61B 5/4528; A61B 2505/09

(57) ABSTRACT

The present invention teaches an articular fracture rehabilitation process measuring device. The measuring device comprises a base plate, a positioning tube, and an electronic angle gauge; the positioning tube and the electronic angle gauge are fixed separately on the base plate. The electronic angle gauge comprises a distance sensor, a rotation angle sensor, a data processor, and a digital display; the data processor is connected to the distance sensor and the rotation angle sensor; the digital display is connected with the data processor.

### FIELD OF THE INVENTION

The present invention generally relates to physical rehabilitation technology. More particularly, the invention is related to an articular fracture rehabilitation process measuring device.

### BACKGROUND OF THE INVENTION

In daily life, fractures may occur to everyone, and it can seriously affect one's daily living activities such as wearing clothes, having a meal, and even walking.

In the rehabilitation stages of fractures, one will need to train the joint part of the fracture every day. Notably, the current rehabilitation training plans and training processes lack a common measurable standard. In response to a patient's inquiry about this, the doctor typically does not have an accurate answer but simply tells the patient to continue exercising regularly. But regarding how much to exercise, there is no exact rule. And the fracture patient cannot learn about the recovery extent in the rehabilitation course, and the intensity and the dynamics of training are unclear. As a result, the patient may not recover as efficiently as it may be and be irritable, therefore deteriorating the rehabilitation progress.

What is needed is an articular fracture rehabilitation process measuring device, which can address the aforementioned issue.

### SUMMARY OF THE INVENTION

The present invention discloses an articular fracture rehabilitation process measuring device.

In one preferred embodiment of the present invention, it discloses an articular fracture rehabilitation process measuring device. The measuring device comprises a base plate, a positioning tube, and an electronic angle gauge; the positioning tube and the electronic angle gauge are fixed separately on the base plate. The electronic angle gauge comprises a distance sensor, a rotation angle sensor, a data processor, and a digital display; the data processor is connected to the distance sensor and the rotation angle sensor; the digital display is connected with the data processor.

A distance between the distance sensor and an exit end of the positioning tube is 10˜25 cm; the distance sensor is used for measuring joint swinging distance; the rotating angle sensor is used for measuring joint rotating angle; the data processor is used for converting the swing distance data to joint swing angle; the digital display is used for displaying the joint swing angle and the joint rotating angle.

In another preferred embodiment, the data processor calculates a joint recovery percentage with the joint swing angle and the joint rotation angle; the joint recovery percentage is displayed on the digital display.

In another preferred embodiment, the articular fracture rehabilitation measuring device further comprises a speaker, wherein the speaker is connected to the data processor; the speaker is used for reporting the joint swinging angle and the joint rotating angle.

In another preferred embodiment, the data processor calculates a joint recovery percentage with the joint swing angle and the joint rotation angle; the joint recovery percentage is reported on the speaker.

In another preferred embodiment, one side of the positioning tube is provided with at least one strengthening gusset; the opposite side is fixed on a side plate; the side plate is fixed vertically on the base plate.

### BRIEF DESCRIPTION OF DRAWINGS AND TABLES

FIG. 1 is a schematic drawing illustrating the front view of an articular fracture rehabilitation process measuring device according to one preferred embodiment of the invention; and

FIG. 2 is a schematic drawing illustrating the top view of an articular fracture rehabilitation process measuring device according to one preferred embodiment of the invention.

### DETAILED DESCRIPTION OF THE INVENTION

The present invention discloses an articular fracture rehabilitation process measuring device. While the present invention may be embodied in many different formulas to prompt an understanding of the principles of the invention, reference will be made to the embodiments illustrated in the drawings, tables, and specific language will be used to describe the same. It will nevertheless be understood that no limitation or restriction of the scope of the invention is thereby intended. Any alterations and further implementations of the principles of the invention as described herein are contemplated as would normally occur to one skilled in the art to which the invention relates.

In one preferred embodiment of the present invention, it discloses an articular fracture rehabilitation process measuring device. The measuring device comprises a base plate, a positioning tube and an electronic angle gauge; the positioning tube and the electronic angle gauge are fixed separately on the base plate. The electronic angle gauge comprises a distance sensor, a rotation angle sensor, a data processor and a digital display; the data processor is connected to the distance sensor and the rotation angle sensor; the digital display is connected with the data processor.

A distance between the distance sensor and an exit end of the positioning tube is 10˜25 cm; the distance sensor is used for measuring joint swinging distance; the rotating angle sensor is used for measuring joint rotating angle; the data processor is used for converting the swing distance data to joint swing angle; the digital display is used for displaying the joint swing angle and the joint rotating angle.

In another preferred embodiment, the data processor calculates a joint recovery percentage with the joint swing angle and the joint rotation angle; the joint recovery percentage is displayed on the digital display.

In another preferred embodiment, the articular fracture rehabilitation measuring device further comprises a speaker, wherein the speaker is connected to the data processor; the speaker is used for reporting the joint swinging angle and the joint rotating angle.

In another preferred embodiment, the data processor calculates a joint recovery percentage with the joint swing angle and the joint rotation angle; the joint recovery percentage is reported on the speaker.

In another preferred embodiment, one side of the positioning tube is provided with at least one strengthening gusset; the opposite side is fixed on a side plate; the side plate is fixed vertically on the base plate.

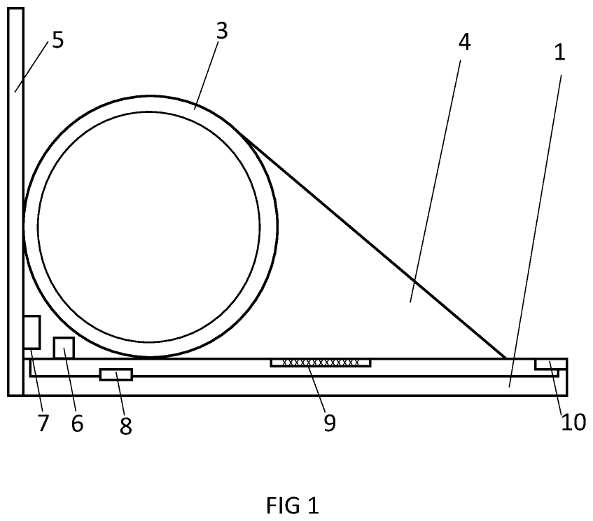

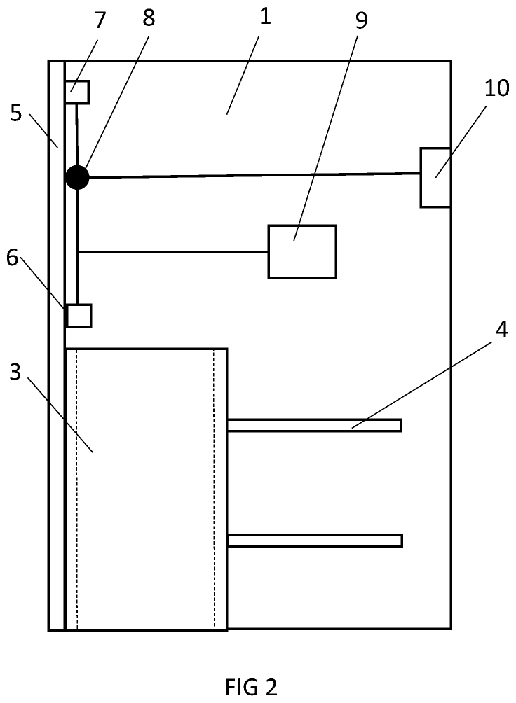

FIG. 1 is a schematic drawing illustrating the front view of an articular fracture rehabilitation process measuring device according to one preferred embodiment of the invention; and FIG. 2 is a schematic drawing illustrating the top view of an articular fracture rehabilitation process measuring device according to one preferred embodiment of the invention.

Referring to FIG. 1 and FIG. 2: the articular fracture rehabilitation process measuring device comprises a base plate 1, a positioning tube 3 and an electronic angle gauge. The positioning tube 3 and the electronic angle gauge are separately fixed at base plate 1. The electronic angle gauge comprises a distance sensor 7, a rotation angle sensor 6, a data processor 8, and a digital display 9. The data processor 8 is connected with the distance sensor 7 and the rotation angle sensor 6, and the digital display 9 is connected with the data processor 8. The distance between the distance sensor 7 and the exit end of the positioning tube 2 exit is 10˜25 cm. The distance sensor 7 is used for measuring joint swinging distance. The data processor 8 is used to calculate the joint swinging angle from the joint swinging distance data. The digital display 9 is used for displaying the joint swinging angle.

When using the measuring device, a patient places the joint part of the fracture through positioning tube 3, which is fixed on base plate 1, and starts to swing the movable joint. When the patient swings to a certain angle and cannot continue to swing further, the distance sensor 7 detects the distance that the joint swing, and transmits the distance data to the data processor 8. The data processor 8 calculates the joint swing angle, and finally, the joint swing angle is shown on the digital display 9, and the patient can record the current joint swing angle.

The following is the formula for calculating the joint swing angle from the joint swing distance:

tan θ=X/Y

Wherein, θ is the joint swing angle; X is the joint swing distance that the distance sensor 7 detects; Y is the distance from the joint swing pivot to the distance sensor 7.

In order to ensure the positioning intensity of positioning tube 3 in the measuring device, one side of the positioning tube 3 of the measuring device is provided with at least one strengthening gusset 4; and the opposite side is fixed to a side plate 5. The side plate 5 is fixed vertically to the base plate 1.

A joint can not only swing but also can rotate to a specific angle. Therefore, measuring the joint rotation angle is also required to determine the joint recovery percentage. When using the measuring device, the patient first places the joint part of the fracture through positioning tube 3, holds the rotation angle sensor 6, and starts to rotate the movable joint. When the joint part cannot rotate any further, the joint rotation angle is detected by the rotation angle sensor 6 and sent to the data processor 8. The data processor 8 then sends the joint rotation angle to the digital display 9, which can be observed by the patient.

The data processor 8 then calculates a joint recovery percentage based on the following algorithm. When the joint swing angle is 0˜30° and the joint rotation angle is −45°˜45°, the joint recovery percentage is approximately 30%; when the joint swing angle is 30°˜45° and the joint rotation angle is −75°˜75°, the joint recovery percentage is approximately 55%; when the joint swing angle is 45°˜60° and the joint rotation angle is −90°˜90°, the joint recovery percentage is 80%; when the joint swing angle is 60°˜80° and the joint rotation angle is −110°˜110°, the joint recovery percentage is approximately 90%; and when the joint swing angle is 80°˜90° and the joint rotation angle is −120°˜120°, the joint recovery percentage is 100%. The data processor 8 then sends the joint recovery percentage to the digital display 9, which the patient can observe.

To make the measuring process more intuitive, a speaker 10 is also connected to the data processor 8. Speaker 10 is used for reporting the current joint swing angle, the joint rotation angle, and the joint recovery percentage. When the joint recovery percentage does not reach certain requirements, speaker 10 can say a language to encourage the patient to continue the exercise. On the other hand, when joint recovery percentage reaches certain requirements, speaker 10 can say some language to let the patient know that the joint has been fully recovered.

By using this measuring device daily, the patient can measure and record the joint swing angle, joint rotation angle, and joint recovery percentage, and these records can truly reflect the entire articular fracture rehabilitation process.

The positioning tube mentioned above is for the purpose of fixing the joint into its place and therefore is not restricted to tubular structure as mentioned herein. In other embodiments, it may be of square tube structure or of a semicircle jacket, which is to be fixed on the base plate to form a semi-cylindrical structure. Furtherly, the positioning tube can be replaced with other fixing mechanisms such as a belt or a notch, which is used to fix the joint position into place.

With the measuring device of the present invention, the rehabilitation situation in patients with articular fracture can be reflected intuitively. The measuring device can be used for the rehabilitation process after any particular fracture. The size of the measuring device can be adjusted to fit the fracturing position during the manufacturing process.

The invention has the following advantages:

1. The present invention will enable a patient to see the progress of rehabilitation daily, thus empowering the patient to joyfully participate in the fracture recovering process.

2. The present invention enables the rehabilitation training of fracture patients to progress with a plan, specified intensity, and order.

3. The present invention is cheap to manufacture with practical usage, thus reducing a patient's financial burden.

4. The present invention can help the head of a family of fracture patients to help children to take exercise within the scope of rational dynamics, recovery limb function as early as possible.

The above detailed description of the articular fracture rehabilitation process measuring device is illustrative but not restrictive. Many more embodiments may be included within the limit of the scope.

Any modifications and adoptions without departing from the scope and spirit as set forth in the claims will still be within the protection of this invention.

I hereby claim:

1. An articular fracture rehabilitation process measuring device, comprising a base plate, a positioning tube and an electronic angle gauge; said positioning tube and said electronic angle gauge are fixed separately on said base plate; said electronic angle gauge comprises a distance sensor, a rotation angle sensor, a data processor and a digital display; said data processor is connected to said distance sensor and said rotation angle sensor; said digital display is connected with said data processor; wherein
a distance between said distance sensor and an exit end of said positioning tube is 10˜25 cm; said distance sensor is used for measuring a joint swinging distance; said rotating angle sensor is used for measuring a joint rotating angle; said data processor is used for converting said joint swing distance data to a joint swing angle; said digital display is used for displaying said joint swing angle and said joint rotating angle.

2. The articular fracture rehabilitation process measuring device of claim 1, wherein said digital processor calculates a joint recovery percentage with said joint swing angle and said joint rotation angle, said joint recovery percentage is displayed on said digital display.

3. The articular fracture rehabilitation process measuring device of claim 1, further comprising a speaker, wherein said speaker is connected to said data processor; said speaker is used for reporting said joint swinging angle and said joint rotating angle.

4. The articular fracture rehabilitation process measuring device of claim 3, wherein said data processor calculates a joint recovery percentage with said joint swing angle and said joint rotation angle, said joint recovery percentage is reported on said speaker.

5. The articular fracture rehabilitation process measuring device of claim 1, wherein one side of said positioning tube is provided with at least one strengthening gusset; the opposite side is fixed on a side plate; said side plate is fixed vertically on said base plate.
